(19) United States
(12) Patent Application Publication
(10) Pub. No.: US 2010/0076473 A1
(43) Pub. Date: Mar. 25, 2010

(54) Tissue Slicer

(76) Inventors: Ossama Tawfik, Leawood, KS (US); Swaran K. Jain, Lansing, KS (US); Maged Fanous, Overland Park, KS (US); Brooke Montgomery, Overland Park, KS (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 12/567,395

(22) Filed: Sep. 25, 2009

Related U.S. Application Data
(60) Provisional application No. 61/100,050, filed on Sep. 25, 2008.

(51) Int. Cl. A61B 17/32

(52) U.S. Cl. 606/167

(57) ABSTRACT

A tissue slicer having a partially open base for permitting the slicing blades to transverse therethrough, a dual pivoting member for activating the sliding blades in a vertical direction, a blade cartridge with a plurality of blades and a multi-pin specimen holder. The device secures the tissue specimen without distortion in place during the slicing process, protect the user while cutting specimens, standardizes tissue sections for optimal processing, improves the quality of sections for microscopic evaluation, and improves diagnostic accuracy and reliability.

### CROSS-REFERENCE TO RELATED APPLICATIONS

This application is based on and claims priority to U.S. Provisional Application Ser. No. 61/100,050 filed on Sep. 25, 2008, which is incorporated herein by reference.

### STATEMENT REGARDING FEDERALLY SPONSORED RESEARCH OR DEVELOPMENT

Not applicable.

### BACKGROUND OF THE INVENTION

1. Field of the Invention

This invention relates to a biological tissue slicer designed to produce thin, uniform slices of tissue suitable for biochemical, pharmacological, toxicological, pathologic, autopsy, animal tissue research studies, and other applications. The invention produces standardized tissue sections of a defined thickness for optimal tissue processing.

2. Description of Related Art

Specimen sampling is one of the most critical steps in achieving the correct diagnosis. Currently, pathologists grossly evaluate surgical tissue specimens and tediously cut the sections with primitive blades, razors, and scalpels slice-by-slice in order to prepare the tissue for analysis and diagnosis. That process sometimes leads to many unwelcomed results, such as cutting injuries to lab personnel, inadequate tissue sampling, insufficient tissue processing, delay in patient care, potential harm to the patient if diagnosis is delayed or is incorrect and a potential increase in health care costs. The specimens differ in sizes and shapes, and typically vary from a minute fragment of tissue measuring less than 1 mm to large complex specimens up to 90 cm or larger. Appropriate sectioning requires slicing the specimens that are typically about 3 to 5 mm thick and then packaging them into tissue cassettes for processing, embedding into paraffin wax, and tissue sectioning to produce microscopic slides.

### BRIEF SUMMARY OF THE INVENTION

The present invention is directed to a biological tissue slicer device which rapidly produces slices of tissue of identical uniform thickness, while minimizing trauma and structural distortion, and contamination to the tissue, thus standardizing tissue sections for optimal processing, improving quality of tissue sections for optimal microscopic evaluation, and ultimately improving diagnostic accuracy and reliability. The tissue slicer eliminates the risk of sharp injury to pathologists, pathology residents, and pathologists' assistants. This device also reduces healthcare costs and increases efficiency by decreasing the time spent cutting specimens manually and/or eliminating injuries. Another beneficial aspect of this invention is the simplicity of the device and cost-effectiveness to fabricate.

In a first exemplary embodiment, the biological tissue slicer comprises a blade cartridge having a plurality of blades, and a base for positioning a tissue specimen thereon. These blades may have the same length or be of variable lengths, and may be positioned at the same or variable distances from one another. Thus, the user is able to quickly, safely, and precisely cut a biologic tissue specimen to a desired thickness.

The base has a plurality of openings or gaps for permitting the plurality of blades to extend through the entire thickness of the tissue specimen (and even beyond the specimen thickness) during the slicing process. The base preferably comprises a plurality of plates (e.g., L-shaped plates) aligned in parallel such that a gap between two adjacent plates forms one of the plurality of openings. The preferred base secures the tissue specimen in an efficient and safe manner, and also helps guide the blades while cutting the specimen both vertically and horizontally, permitting standardization of the tissue section thicknesses quickly and safely.

In one aspect, the tissue slicer includes one or more vertical guide rods attached to the base. The vertical guide rods are adapted to slidably and removably engage a multi-pin specimen holder comprising a pin base and a plurality of pins. The vertical guide rods are movably engaged with one or more horizontal guide rods attached to the base. The horizontal guide rods are adapted to guide the vertical guide rods in a horizontal direction to position the multi-pin specimen holder in the desired location on the base. A U-channel may be used to connect the vertical guide rods to the horizontal guide rods. By turning a threaded rod which is attached to the U-channel, the vertical guide rods are moved in a horizontal direction as desired by the user. Thus, the user is able to secure the specimen in position without deforming, destroying, or damaging the specimen. Further, the guide rods improve safety by minimizing the risk for hand injury.

In still another aspect, the biological tissue slicer of the present invention comprises a multi-pin specimen holder having plurality of pins extending from a pin base. The multi-pin specimen holder is preferably removably attached to the vertical guide rods. In one aspect, a single row of pins is provided on a multi-pin specimen holder. The pins may be arranged in any suitable configuration. In another aspect, the pins are arranged in one or more rows of pins, such that the pins in one row are spaced at the same or different distance than the pins in another row. The pins may or may not have the same diameter or length. Thus, the user is able to secure the specimen in position without deforming, destroying, or damaging the specimen. Further, the multi-pin specimen holder improves safety by minimizing the risk for hand injury. Further, the multi-pin specimen holder provides flexibility and efficiency in cutting samples at variable thicknesses at a low cost.

In still another aspect, a plurality of pins extend from the base of the tissue slicer, and the pins are pivotably attached thereto using a pivot rod, lever, and spring assembly. The pins are preferably J-shaped or U-shaped. When the user actuates the lever, the lever causes the pins to pivot about the pivot rod. The user may then position the biological tissue specimen on the base. When the lever is released, the spring biases the lever such that the pins engage the biological tissue specimen. Once the tissue specimen is placed on the base, one or more secondary pins may be positioned through the holes in the pivoting pins to further engage and stabilize the tissue specimen.

In yet another aspect, the biological tissue slicer of the present invention includes a blade cartridge that is attached to a reciprocating driver, such as one found in commercially available electrical devices or ultrasonic devices. During use, the user moves the blades through the tissue specimen and into the openings or gaps on the base, thereby enabling the user to cut through the entire thickness of the specimen. The multi-pin specimen holder permits the user to position and stabilize the tissue specimen in the desired position and also serve as a guide for the blades during the cutting process. Thus, the user is able to quickly, safely, and precisely cut a biological tissue specimen using the tissue slicer system of the present invention.

In another embodiment, the biological tissue slicer base comprises a pin base having a plurality of pins extending therefrom. The pins are configured to form a plurality of openings for permitting the blades to extend therethrough. Thus, the blades are capable of extending through the entire tissue specimen (and even beyond the tissue specimen) during the slicing process. The pin base is preferably removable from the base so that the pin configuration, pin diameter, pin length, or combinations thereof can be adjusted by the user as desired for a given biological tissue specimen. The pin base thus permits the user to position and stabilize the tissue specimen in the desired position without structural distortion or damage to the tissue specimen.

In another aspect, the biological tissue slicer includes a blade cartridge with a plurality of blades. The blade cartridge is positioned in a cartridge plate that is connected to a dual pivoting member. The dual pivoting member is pivotable about a first fixed pivot axis and a second movable pivot axis. The blades are thus adapted to cut a tissue specimen by moving the blades both vertically and horizontally to mimic a slicing action. The blades may be removable from the blade cartridge. The blade cartridge may also be removable from a blade cartridge plate which is attached to the dual pivoting member. The blades may be of the same or variable lengths and may be positioned at the same or variable distances from one another. Thus, the user is able to quickly, safely, and precisely cut a biologic tissue specimen.

In yet another aspect, the tissue slicer includes two vertical members extending from the base and a transverse horizontal bar extending between the two vertical members. The dual pivoting member is pivotally and fixedly connected to the transverse bar, which forms the first fixed pivot axis. This permits the user to simulate a slicing action in serially sectioning a tissue specimen without structural distortion.

In still another aspect, the dual pivoting member is pivotably and movably connected to the blade cartridge via one or more holding brackets attached to the cartridge plate. In a preferred aspect, a connecting rod extends between two holding brackets. Thus, the dual pivoting member is pivotably and movably connected to the connecting rod, which forms the second movable axis. This permits the user to simulate a slicing action in serially sectioning a tissue specimen without structural distortion.

The brief description that follows will reveal additional aspects of the invention as well as advantages and novel features. The usefulness of the invention will be readily apparent to those skilled in the art of gross examination and dissection or may be learned from using this invention. The objectives and advantages of the invention may be realized and attained by means of the instrumentalities and combinations particularly pointed out in the appended claims.

### BRIEF DESCRIPTION OF THE DRAWINGS

FIG. 1 is a perspective view of a base and multi-pin specimen holder of a tissue slicer in accordance with a first embodiment of the present invention.

FIG. 2 is a side view of the base and multi-pin specimen holder of the tissue slicer shown in FIG. 1. One of the horizontal guide rods has been removed so that the threaded rod connected to the U-channel is visible.

FIG. 3 is a front view of the base and multi-pin specimen holder of the tissue slicer shown in FIG. 1.

FIG. 4 is a perspective view of a blade cartridge positioned in a holding bracket attached to an exemplary commercially available reciprocating device for use with the tissue slicer shown in FIG. 1.

FIG. 5 is a perspective view of the blade cartridge positioned in a holding bracket as shown in FIG. 4.

FIG. 6A is a side view of the blade cartridge of the tissue slicer for use with the holding bracket as shown in FIG. 4.

FIG. 6B is a top view of the cutting blade cartridge shown in FIG. 6A.

FIG. 7A is a perspective view of the multi-pin specimen holder shown in FIG. 1.

FIG. 7B is a perspective view of an alternative multi-pin specimen holder for use in first embodiment or as a stand-alone device.

FIG. 8 is a perspective view of a tissue slicer in accordance with a second embodiment of the present invention. The device is shown with the blade cartridge in a first upper position.

FIG. 9 is another perspective view of the tissue slicer shown in FIG. 8. The device is shown with the blade cartridge in a second lower position.

FIG. 10 is a side view of the tissue slicer shown in FIG. 8.

FIG. 11 a top view of the tissue slicer shown in FIG. 8.

FIG. 12 is a perspective view of a tissue slicer in accordance with a third embodiment of the present invention. The device is shown with the blade cartridge in a first down position.

FIG. 13 is another perspective view of the tissue slicer shown in FIG. 12. The device is shown with the blade cartridge in a second upper position. In this position, the user is able to position a biological tissue specimen between the pins and the base.

FIG. 14A is a side view of the tissue slicer shown in FIG. 12.

FIG. 14B is a side view of a modified tissue slicer shown in FIG. 12. In FIG. 14B, the base is slidably engaged with the frame using a rail assembly.

FIG. 15 is a perspective view of the blade cartridge and mounting plate for use with the third embodiment of the present invention.

FIG. 16A is a side view of the blade cartridge and mounting plate shown in FIG. 15.

FIG. 16B is a top view of the cutting blade cartridge and mounting plate shown in FIG. 15.

### DETAILED DESCRIPTION OF PREFERRED EMBODIMENT

The present invention is directed to a tissue slicer for slicing biological tissue specimens to a predetermined thickness (e.g., 1, 2, 3, 4, 5 mm thick specimens, or as needed). The tissue slicer may be used in conjunction with various surgical specimens, such as the breast, liver, lung, brain, and kidneys. Furthermore, the tissue slicer is suitable for autopsy, biochemical, pharmacological, toxicological, and animal tissue clinical and research studies.

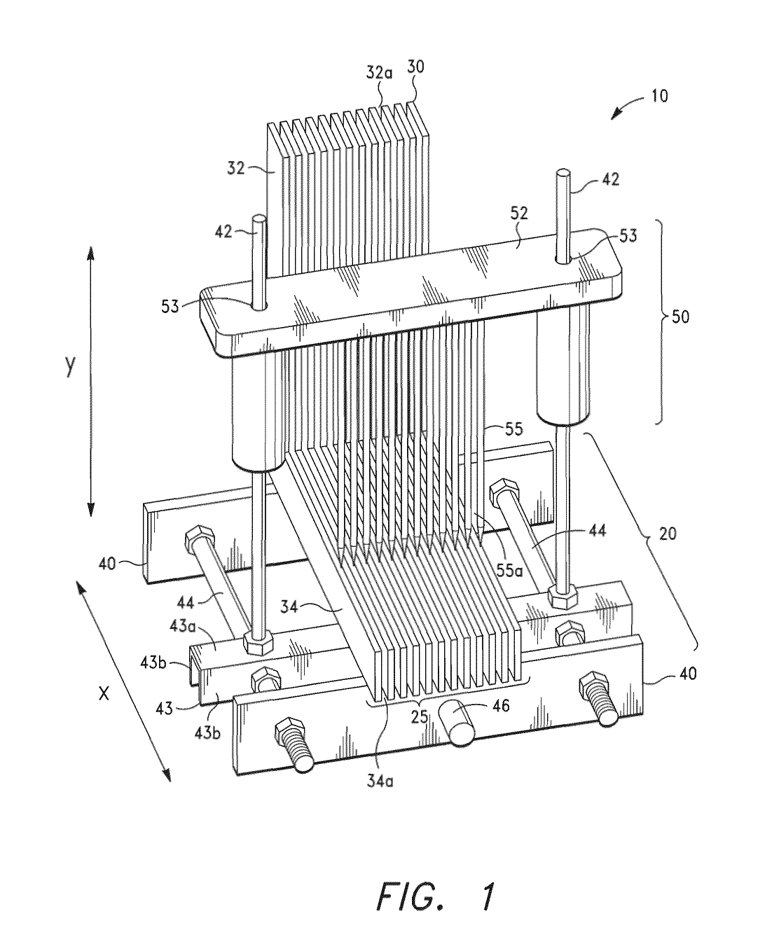

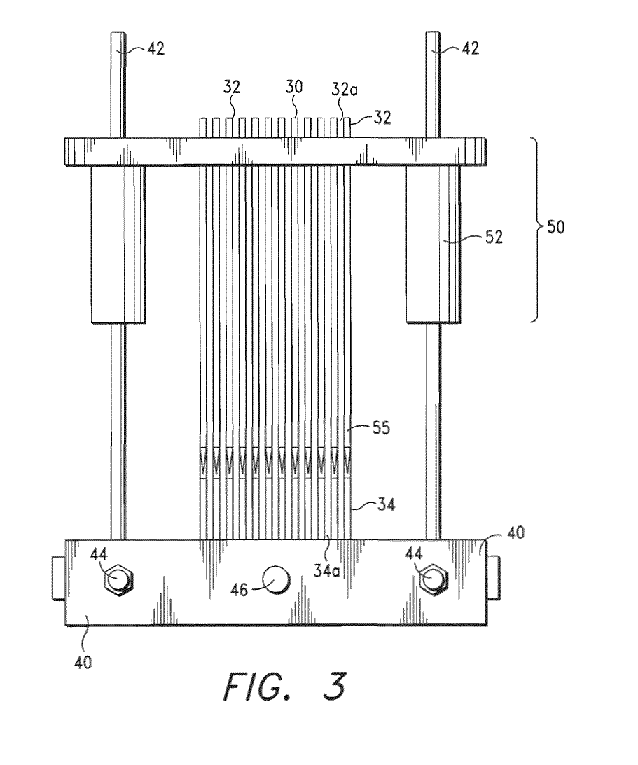

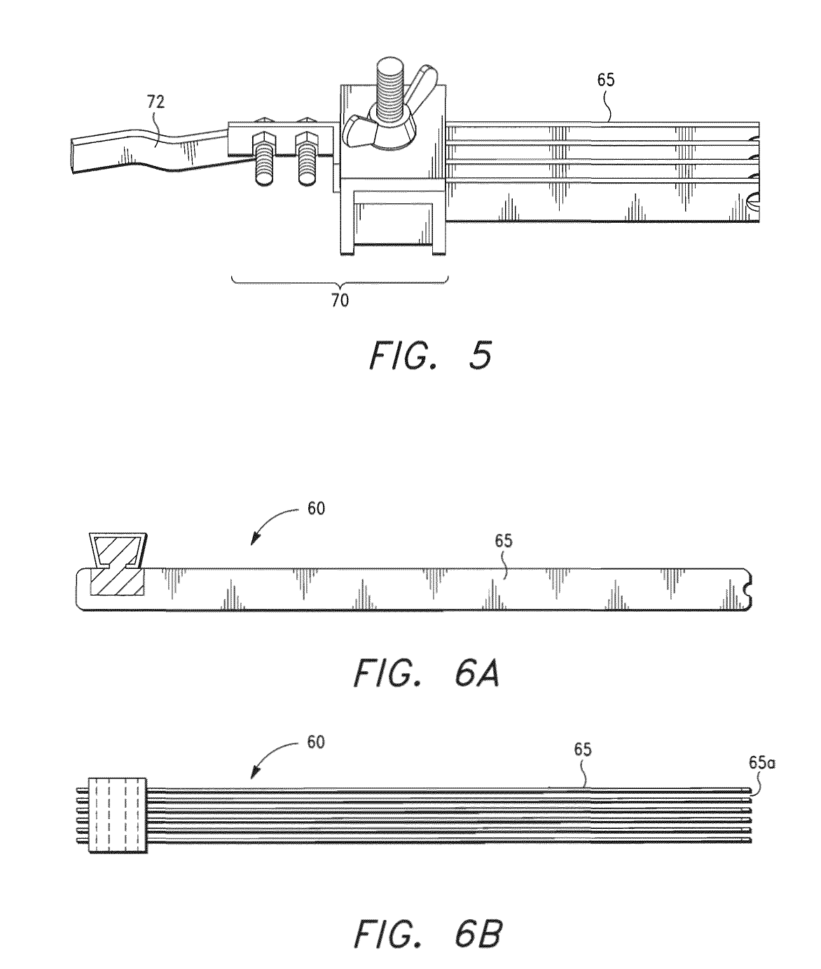

FIGS. 1 to 7B illustrate a tissue slicer 10 in accordance with a first embodiment of the present invention. As shown in FIG. 1, the tissue slicer 10 comprises a base 20 having a partially open horizontal surface 25. The partially open surface 25 has a plurality of openings or slits that permit one or more cutting blade(s) to traverse therethrough. The base 20 is comprised of a plurality of plates 30, preferably angled plates 30, which are aligned to create the openings. The angled plates are aligned in parallel such that there is a gap between the adjacent plates 30. The angled plates 30 preferably comprise metal L-shaped plates, each having a vertical member 32 and a horizontal member 34 spaced about 1 to 10 mm apart (e.g., 1, 2, 3, 4, 5, 6, 7, 8, 9, 10 mm apart, or as needed). The gaps 32a between the vertical members 32 are adapted to receive the blade(s) in the tissue slicer and also function as a guide for precise cutting. The biological tissue specimen may also be positioned against the vertical members 32 to help hold it in place during the slicing process. The horizontal members 34 function as a partially open horizontal surface 25 where the tissue specimen is positioned. The gaps 34a between the horizontal members 34 are adapted to receive the blades(s) in the tissue slicer so that the blade(s) can extend through and cut the entire tissue specimen (and even beyond the thickness of the tissue specimen). The angled plates 30 are mounted on a frame comprising one or more base bars or base plates 40 to maintain the angled plates 30 in a fixed position.

The tissue slicer 10 further comprises a multi-pin specimen holder 50 comprising a pin base 52 and a plurality of pins 55 having gaps 55a therebetween for holding the tissue specimen in place during the slicing process. The plurality of pins 55 are attached to the pin base 52 in any suitable configuration. FIGS. 1, 3, and 7A illustrate the plurality of pins in linear configuration; however, the pins may be configured in other geometric and non-geometric configurations. For example, the pins may be arranged in one or more rows. As another example, the pins may be spaced either equidistantly (e.g., about 2 to 6 mm, preferably about 4 mm apart) or non-equidistantly relative to one another. Typically the pins are about 1 cm to 24 cm long, more preferably about 6 cm to 18 cm long, and most preferably about 12 cm long. The pins are typically about 1 mm to 6 mm in diameter, with a 2 mm diameter being most preferred. The pins are preferably pointed where they engage the tissue specimen, but need not be. During use, the pins 55 preferably engage the top surface of the tissue specimen, and may extend partially through the tissue specimen, or will pass through the tissue specimen to help secure the specimen in place during the tissue slicing process. The pin base is manufactured with any suitable material, such as plastic or metal. The pins are preferably made of metal, such as stainless steel.

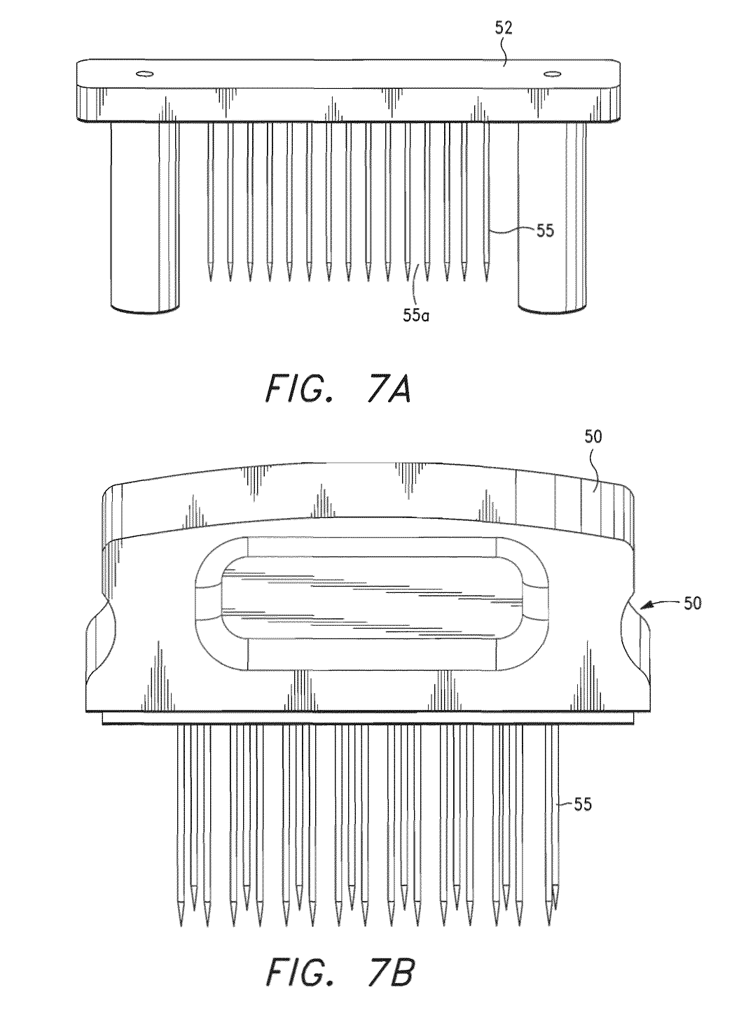

FIG. 7B illustrates an alternative multi-pin specimen holder 50 in for use with the first exemplary embodiment. The holder 50 is essentially the same as that shown in FIG. 7A, except that the pins 55 are arranged in two rows. In the first row, the pins are spaced about 3-4 mm apart. In the second row, the pins are spaced about 7-8 mm apart. The second row of pins provides stabilization for the slicing blades 65 while they are moving up and down along axis y (generally shown in FIG. 1) and/or back and forth along axis x (generally shown in FIG. 1) to slice the biological tissue specimen. The first and second rows of pins are positioned about 1 to 3 cm apart, or as needed. It will be appreciated that FIG. 7B illustrates that the pins are preferably the same length such that both rows are likely to engage the biological tissue specimen, but the blade-stabilizing row of pins may be shorter in length. If the pins are the same length, this provides more leverage and stabilization of the specimen during the slicing process because both rows of pins engage the specimen. Further, because the distance between the pins in one row is different from the other row, the orientation of the multi-pin specimen holder relative to the cutting tool may be reversed, depending on the thickness of the sliced tissue desired. Thus, in the example given, tissue sections about 7-8 mm thick may be prepared by orienting the multi-pin specimen holder in one configuration (i.e., so that the cutting blades traverse the 7-8 mm gaps) while tissue sections about 3-4 mm thick may be prepared by orienting the multi-pin specimen holder in an opposite configuration (i.e., so that the cutting blades traverse the 3-4 mm gaps).

As shown in FIGS. 1 to 3, the pin base 52 is movably attached to one or more vertical guide rods 42. The pin base 52 of the multi-pin specimen holder 50 is slidably engaged with the vertical guide rods 42 to move the pin base 52 in a vertical direction (up and down), which is generally denoted by axis y in FIG. 1. More specifically, openings 53 are provided in the pin base 52 for slidably engaging the vertical guide rods 42.

The multi-pin specimen holder 50 is also preferably removably attached to the one or more vertical guide rods 42. Thus, the multi-pin specimen holder 50 can be used as a stand-alone device, for example for securing a tissue specimen in place on a cutting board while being sliced manually. In addition, the multi-pin specimen holder 50 may be removed from the vertical guide rods 42 and gripped by the user to hold the tissue specimen in place using the pins 55 when the tissue specimen positioned on the partially open horizontal surface 25 of the base 20. That is, the operator may use the multi-pin specimen holder 50 without slidably engaging the vertical guide rods 42.

The vertical guide rods 42 are movably attached to one or more horizontal guide rods 44 connected to the base plates 40 securing the partially open surface 25. The vertical guide 42 rods are slidably engaged with the horizontal guide rods 44 to move the vertical guide rods (and the multi-pin specimen holder 50) in a horizontal direction generally denoted by axis x in FIG. 1. In one aspect, the vertical guide rods 42 are welded or otherwise fastened (bolts, screws, adhesive, etc.) to a connecting rod, such as a U-channel 43 having corresponding openings for slidably engaging the horizontal guide rods 44. The U-channel 43 has a top surface 43a which is fixedly connected to the vertical guide rods 42, and has two side surfaces 43b which slidably engage the horizontal guide rods 44.

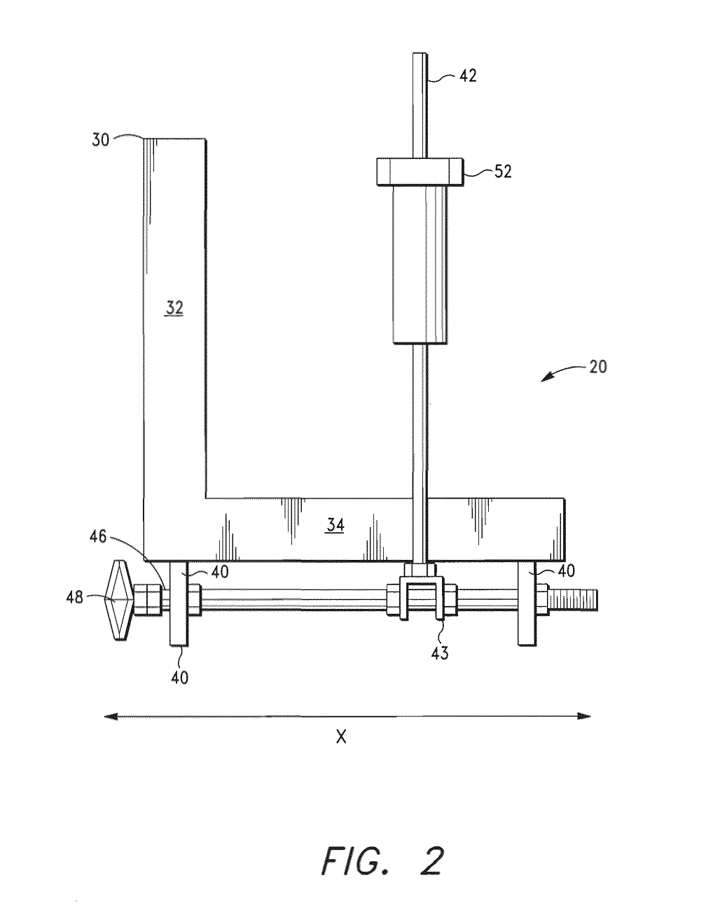

As generally shown in FIG. 2, a threaded rod 46 is preferably disposed through the base plate(s) 40 and is fixedly connected to the U-channel 43. The threaded rod 43 is preferably positioned between the horizontal guide rods 44, and is preferably centered between the horizontal guide rods 44. By moving the threaded rod 46 in a first (e.g., clockwise) direction using a handle 48, the U-channel (and thus the vertical guide rods 42 and multi-pin specimen holder 50) move in a first direction, for example toward the vertical members 32 of the platform base 20. By turning the threaded rod in a second direction (e.g., counter-clockwise), the U-channel (and thus the vertical guide rods 42 and multi-pin specimen holder 50) move in the opposite second direction, for example away from the vertical members 32 of the platform base 20. Thus, by moving the vertical guide rods 42 using the threaded rod 46, the pins 55 may engage and hold the tissue specimen anywhere along the x-axis in the horizontal plane of the platform base 20.

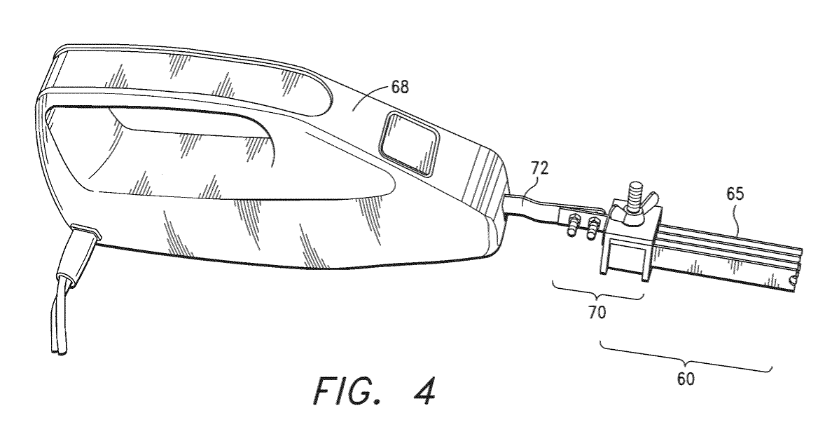

Turning now to FIGS. 4 to 6B, the tissue slicer system 10 of the present invention also includes a blade holder or cartridge 60 having a plurality of blades 65 spaced with gaps 65a therebetween. The size of the gaps 65a preferably correspond to the size of the gaps 32a, 34a between the vertical members 32 and horizontal members 34 of the base 20 so that the blades 65 are capable of cutting the entire thickness of the tissue specimen. Likewise, the size of the gaps 65a between the blades 65 are such that the blades 65 are capable of being moved in the gaps 55a between the pins 55 of the multi-pin specimen holder 50. Thus, the blade cartridge 60 typically positions the blades about 1 to 10 mm apart (e.g., 1, 2, 3, 4, 5, 6, 7, 8, 9, 10 mm apart, or as needed). The blade holder 60 is preferably driven by a reciprocating driver 68, such as those typically made and sold for powering electric or ultrasonic devices, such as electric knives or toothbrushes (see FIG. 4). The blade cartridge 60 is inserted into a holding bracket 70 to permit the release of the blade cartridge 60 from the driver 68 without any additional tools. The cartridge holding bracket 70 is attached (e.g., welded) to the reciprocating driver via a mounting plate 72.

During use, the user moves the blades 65 (which are preferably attached to the reciprocating driver 68) through the gaps 55a between the pins 55 of the multi-pin specimen holder 50, the gaps 34a between the horizontal members 34, and/or the gaps 32a between the vertical members 32 in order to cut the biological tissue specimen. The partially open horizontal surface 25 created by the horizontal members 34 permits the blades to cut through the entire specimen, and even extend below the specimen. Moreover the pins 55 help stabilize the tissue specimen in the desired position, and also serve as a guide for the cutting blades 65. The vertical members 32 help stabilize the specimen in a desired position when the specimen is placed against the vertical members, and may also help service as a guide for the cutting blades 65. Thus, the user is able to quickly and precisely cut a biological tissue specimen using the tissue slicer system of the present invention.

While the blade cartridge 60 is preferably used in conjunction with the reciprocating driver, base, and multi-pin specimen holder as discussed above, the blade cartridge may 60 also be as a separate cutting instrument. That is, the user could manually create the slicing action using the blade cartridge 60 to cut a biological tissue specimen. Further, the blade cartridge 60 could be used with along with the reciprocating driver 68 to cut a biological tissue specimen without the aid of the base and/or multi-pin specimen holder. Lastly, the blade cartridge 60 is well adapted to be used on conjunction with the tissue slicer of the third embodiment (described below).

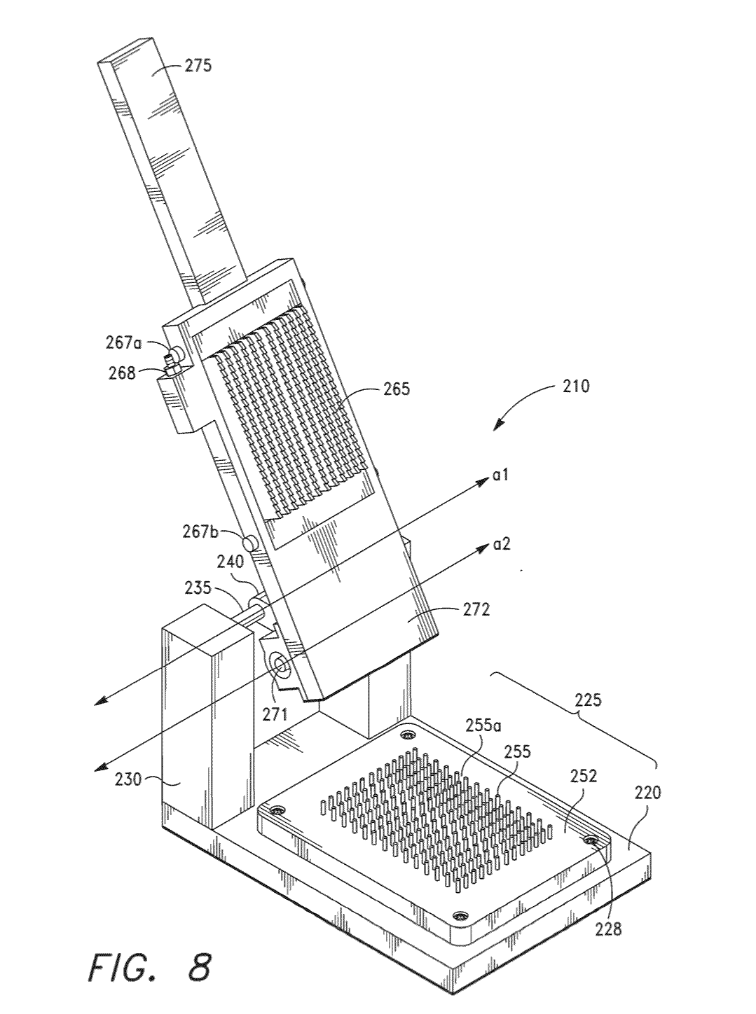

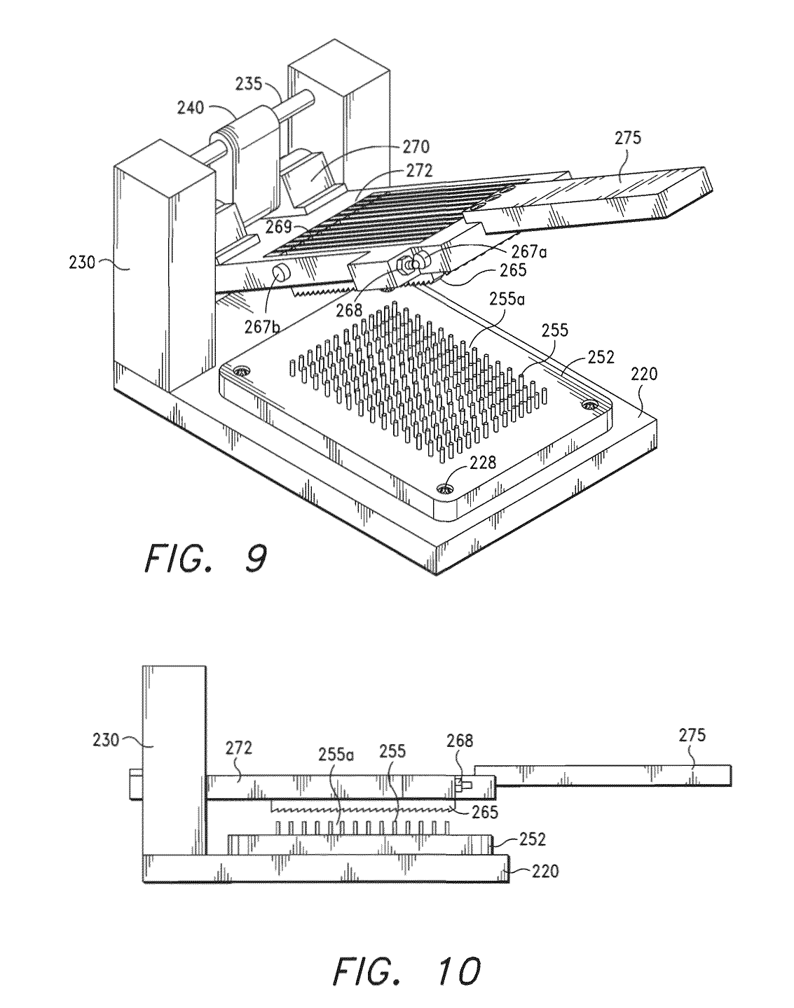

FIGS. 8 to 11 illustrate a biological tissue slicer 210 in accordance with a second embodiment of the present invention. The tissue slicer 210 comprises a base plate 220 and at least one vertical member 230 for engaging a dual pivoting member 240 connected to a blade cartridge 260 (as discussed more fully below). The base plate 220 is adapted to receive a removable pin base 252 holding a plurality of pins 255 that forms a partially open horizontal surface 225. The pins are preferably pointed where they engage the tissue specimen, but need not be as shown in the figures. FIG. 8 illustrates the plurality of pins 255 in a linear rectangular configuration; however, the pins may be configured in other geometric and non-geometric configurations. In addition, the pins may be spaced either equidistantly (e.g., about 2 to 6 mm, preferably about 4 mm) or non-equidistantly relative to one another. However, the pins are positioned such that there are gaps 255a that permit the cutting blades 265 in the blade cartridge 260 to extend through the bottom surface of the tissue specimen and reach the pin base 252 as discussed below. Typically, the pins 255 are about 0.3 cm to 2 cm long, more preferably about 0.5 cm to 1.0 cm long. The pins 255 are typically about 0.5 mm to 3 mm in diameter, with a 1 mm diameter being most preferred. The pin base 252 is removably attached to the base plate 220 via one or more screws or bolts 228.

In a preferred aspect, two vertical members 230 extend from the base plate 220. A transverse horizontal bar 235 extends between the two vertical members 230 and forms a first fixed pivot axis as generally denoted by axis a1 in FIG. 8. A dual pivoting member 240 extends from the transverse bar 235 and is fixedly pivotable about the first fixed pivot axis a1 defined by the transverse bar 235. The dual pivoting member 240 is also connected to a rod 271, which forms a second movable pivot axis as generally denoted by axis a2 in FIG. 8. The rod 271 extends between two holding brackets 270. The holding bracket 270 is attached to cartridge plate 272, which has an opening for receiving a blade cartridge 260. Thus, the dual pivoting member 240 permits the cartridge plate 272 to be moved both vertically and horizontally to form a “slicing” action.

The blade cartridge 260 has a plurality of cutting blades 265. The individual blades 265 may be removed and replaced from the blade cartridge as needed. Moreover, the entire blade cartridge 260 may be removed and replaced from the cartridge plate 272 as needed. As shown in FIG. 11, the blade cartridge 260 preferably has two transverse holes at each end for engaging two corresponding rods 267a/b that hold both ends of the blade cartridge 260 in a fixed position in the cartridge plate 272. Thus, the blade cartridge 260 may be removed from the cartridge plate 272 by removing the rods 267a/b. The blade cartridge 260 is constructed so that rod 267a also extends through the blades 65. A blade tightening means comprising a screw, bolt, or nut 268 is also positioned adjacent to rod 267a. By moving the tightening means 268 outward (i.e., toward handle 275), the tightening means engages and moves the rod 267a outward as well, thereby tightening the blades 265 within the blade cartridge 260.

The blades 265 in the blade cartridge 260 are spaced at a distance 265a corresponding to the gaps 255a between pins 255 in the pin base using spacers between blades 265. Thus, the blade cartridge 260 typically positions the blades about 1 to 10 mm apart (e.g., 1, 2, 3, 4, 5, 6, 7, 8, 9, and 10 mm apart, or as needed). The blades 265 may be of the same or variable length. Further, the size and number of the blades may vary, providing the user options for slicing various types of specimens.

During use, the user grips onto the handle 275 which causes the blade cartridge 260 to fixedly pivot about the fixed pivot axis a1 and movably pivot about the movable pivot axis a2 via the dual pivoting member. The dual pivoting member is pivotally and fixedly connected to the transverse bar 235, which forms the first fixed pivot axis. Thus, the dual pivoting member is pivotably and movably connected to the connecting rod 271, which forms the second movable axis. The blades 265 thus engage and cut the tissue specimen in a slicing action, moving both vertically and horizontally.

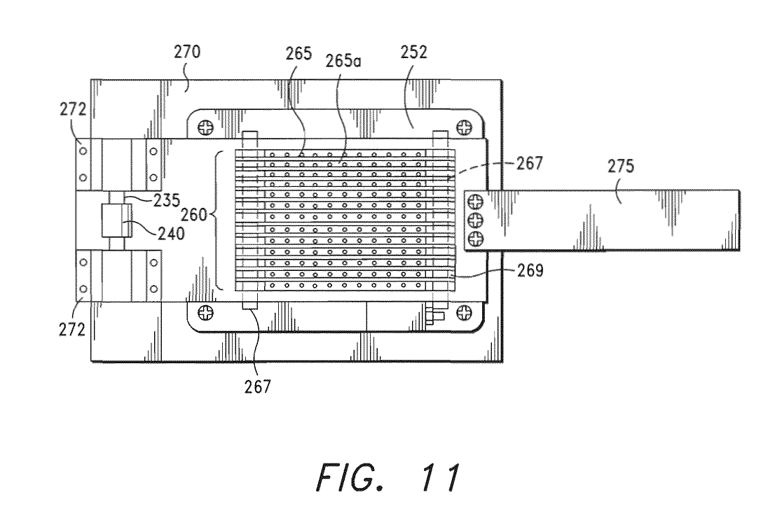

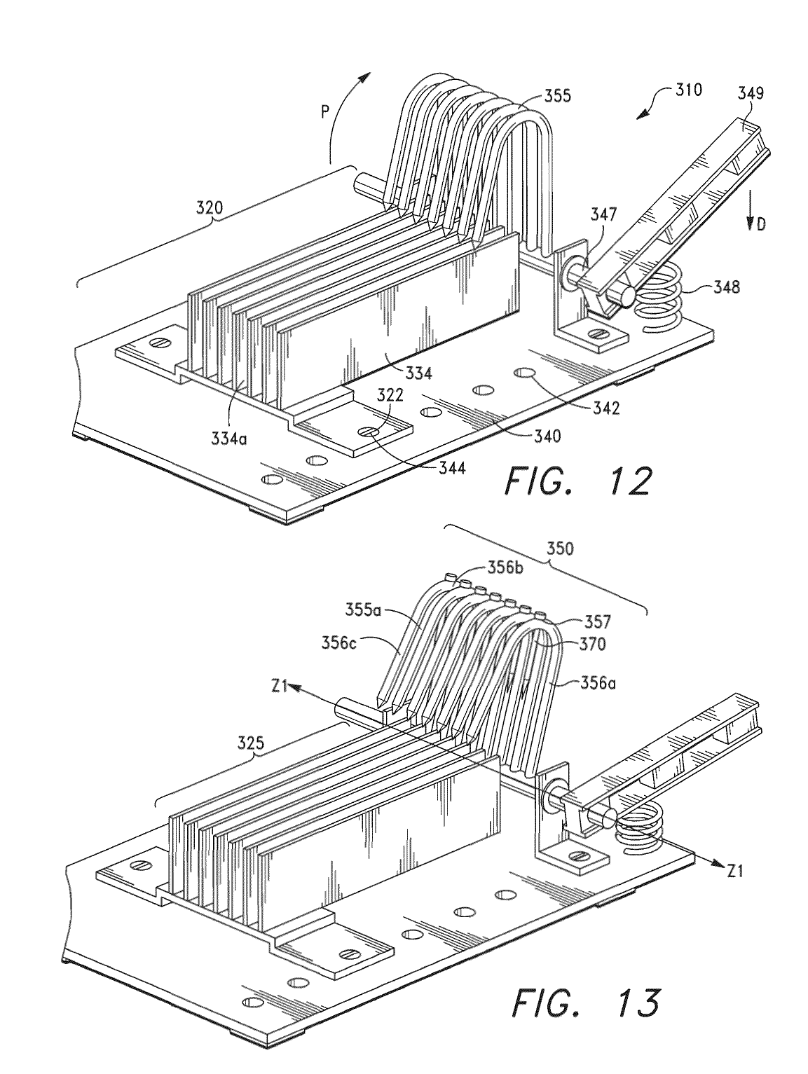

FIGS. 12 to 16B illustrate a tissue slicer 310 in accordance with a third embodiment of the present invention. As shown in FIGS. 12 to 14A, the tissue slicer 310 comprises a base 320 having a partially open horizontal surface 325. The base 320 is comprised of a plurality of horizontal plate members 334 spaced about 1 to 10 mm apart (e.g., 1, 2, 3, 4, 5, 6, 7, 8, 9, 10 mm apart, or as needed). The horizontal plate members 334 function as a partially open horizontal surface 325 where the tissue specimen is positioned. The gaps 334a between the horizontal plate members 34 are adapted to receive the blades(s) in the tissue slicer so that the blade(s) can extend through and cut the entire tissue specimen (and even beyond the thickness of the tissue specimen).

The base 320 having the horizontal plates 334 is mounted to a frame 340 to maintain the horizontal plate members 334 in an adjustable position. The base 320 includes a means for slidably moving the base 320 relative to the pins 355 along the frame 340. As shown in FIGS. 12 to 14A, the base 320 contains at least one hole 322 and the frame 340 contains a plurality of holes 342 receiving an adjustment fastener 344. The adjustment fastener 344 extends through a hole 322 and one of the holes 342 in order to adjust the position of the base 320 relative to the pins 355. Suitable fasteners include bolts, screws, and the like. It will also be appreciated that the base 320 can readily be removed from the frame 340 so that the user may substitute different bases 320 having horizontal plate members 334 of different lengths and/or gaps 334a.

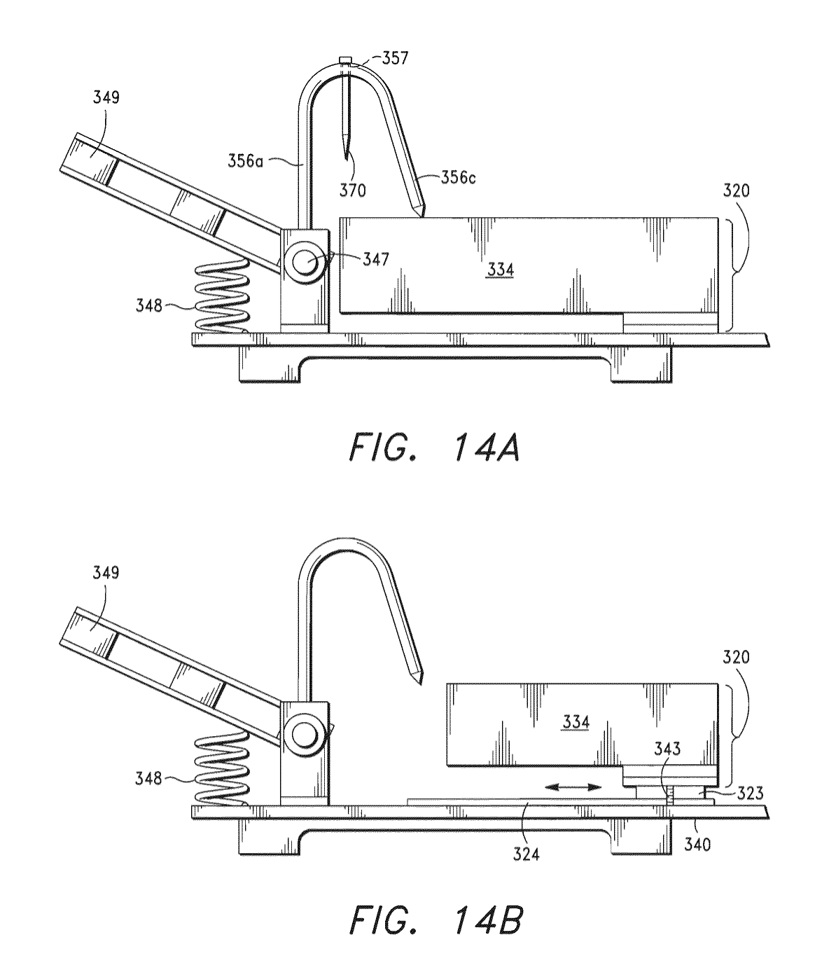

An alternative means for slidably moving the base 320 along the frame 340 is shown in FIG. 14B, and generally comprises a rail assembly. The base 320 may include an upper rail 323 which is slidably engaged with a lower rail 324 positioned on frame 340. The base 320 (and horizontal plates 334) are positioned anywhere along the rail 324 using a latch, fastener, or other locking means 343 as is known in the art.

A pivoting multi-pin specimen holder 350 extends from the frame 340. The pivoting multi-pin specimen holder includes a plurality of pins 355 having gaps 355a therebetween. The pins 355 are preferably J-shaped or U-shaped as generally shown in the drawings. More specifically, each pin has a first vertical section 356a, a top section 356b having an optional hole 357 therein, and a second vertical section 356c for engaging the biological issue specimen. The pins 355 are attached to a pivot rod 347 connected to the base 340. The pins 355 are pivotable about a first fixed pivot axis z1 defined by the pivot rod 347. A spring 348 is used to bias a lever 349 used to pivot the multi-pin specimen 350 holder about axis z1.

When the user actuates the lever 349 by pressing the lever 349 in a general downward motion as shown by arrow D (see FIG. 12), the lever 349 causes the multi-pin specimen holder 350 to pivot about pivot axis z1 in the direction generally shown by arrow P (see FIG. 12). The pins are raised as generally shown in FIG. 13, and the user may then position the biological tissue specimen on the base 320 having a partially open horizontal surface 325. When the lever 349 is released, the spring biases the lever 349 such that the pivoting multi-pin specimen holder 350 engages the biological tissue specimen. For this biasing, the spring 348 has one end attached to the frame 340 and the other end attached to the lever 349.

Once the tissue specimen is placed on the base, one or more secondary pins 370 may optionally be positioned through the holes 357 of the pivoting pins 355 to further engage and stabilize the tissue specimen. In a preferred aspect, the pins 55 in the multi-pin-specimen holders 50 shown in FIGS. 7A and 7B may be used as secondary pins 370. In such a case, the pivoting pins 355 are preferably spaced at a distance corresponding to pins in the multi-pin specimen holder 50 so that each pivoting pin 355 may slidably engage a pin 50 from the multi-pin specimen holder 50 in a corresponding hole 357.

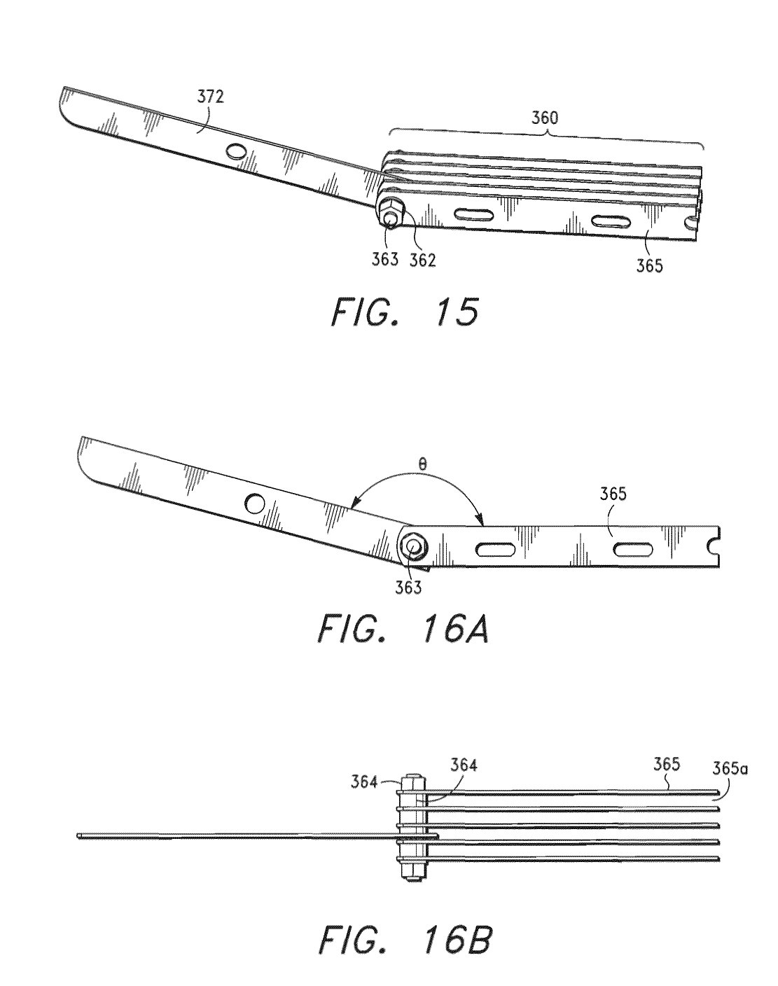

Turning now to FIGS. 15-16B, the tissue slicer system 310 of the present invention also includes a blade holder or cartridge 360 having a plurality of blades 365 spaced with gaps 365a therebetween. The size of the gaps 365a preferably correspond to the size of the gaps 334a between the horizontal members 334 of the base 320 so that the blades 365 are capable of cutting the entire thickness of the tissue specimen. Likewise, the size of the gaps 365a between the blades 365 are preferably such that the blades 365 are capable of being moved in the gaps 355a between the J-shaped or U-shaped pins 355. Thus, the blade cartridges 360 typically position the blades about 1 to 10 mm apart (e.g., 1, 2, 3, 4, 5, 6, 7, 8, 9, 10 mm apart, or as needed). The blade cartridge 360 is preferably driven by a reciprocating driver, such as those typically made and sold for powering electric knives or ultrasonic toothbrushes (see FIG. 4).

The blade cartridge 360 is connected to the reciprocating driver via a mounting plate 372. The blades 365 each contain a hole 362 for receiving a bolt 363. Likewise the mounting plate 372 includes a hole (not shown) for receiving a bolt 363. The mounting plate 372 and the blades 365 are preferably angled to form an angle θ. The angle θ preferably ranges between 150 and 180 degrees. Metal or plastic spacers 364 are used to separate the blades 365 along the bolt 363. A locking nut 364 is tightened at one end to secure the blades 365 and the mounting plate 372 on the bolt 363. A hardening adhesive or plastic mold (not shown) is then preferably applied over the bolt/blade/mounting plate assembly. Together, the blade cartridge and the mounting plate form a one-piece unit which is placed into the reciprocating driver using the mounting plate 372.

During use, the user moves the blades 365 (which are preferably attached to the reciprocating driver) through the gaps 355a between the pins 355 of and/or the gaps 334a between the horizontal plates 334 in order to cut the biological tissue specimen. The partially open horizontal surface 325 created by the horizontal members 334 permits the blades 365 to cut through the entire specimen, and even extend below the specimen. Moreover the pins 355 (and optional secondary pins 370) help stabilize the tissue specimen in the desired position, and also serve as a guide for the cutting blades 365. The first vertical section 356a of the pins 355 help stabilize the specimen in a desired position when the specimen is placed against the vertical section 356a, and may also help service as a guide for the cutting blades 365. Thus, the user is able to quickly and precisely cut a biological tissue specimen using the tissue slicer system of the present invention.

While the blade cartridge 360 is preferably used in conjunction with the reciprocating driver, base, and pins as discussed above, the blade cartridge may 360 also be as a separate cutting instrument. That is, the user could manually create the slicing action using the blade cartridge 360 to cut a biological tissue specimen. Further, the blade cartridge 360 could be used with along with the reciprocating driver to cut a biological tissue specimen without the aid of the base or the pins. Lastly, the blade cartridge 360 is well adapted to be used on conjunction with the tissue slicer of the first embodiment (described above).

From the foregoing, it will be seen that this invention is one well adapted to attain all ends and objectives herein above set forth, together with the other advantages which are obvious and which are inherent to the invention. Since many possible embodiments may be made of the invention without departing from the scope thereof, it is to be understood that all matters herein set forth or shown in the accompanying drawings are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. A biological tissue slicer system for slicing a biological tissue specimen comprising:
a blade cartridge having a plurality of blades;
a base for positioning a tissue specimen, said base having a plurality of openings for permitting the plurality of blades to extend through the entire thickness of said tissue specimen.

2. The biological tissue slicer system of claim 1 wherein said base comprises a plurality of plates aligned in parallel such that a gap between two of said plurality of plates forms one of said plurality of openings for permitting a blade of said plurality of blades to extend therethrough.

3. The biological tissue slicer system of claim 2 wherein said plurality of plates comprises a plurality of L-shaped plates, each L-shaped plate having a vertical member and a horizontal member, and when said plates are spaced apart in parallel to form said plurality of openings.

4. The biological tissue slicer system of claim 2 wherein said plurality of plates are mounted to a base to maintain the plates in a fixed position, and wherein said base has an upper rail for moving said base and said plurality of plates in a horizontal direction.

5. The biological tissue slicer system of claim 1 further comprising one or more vertical guide rods attached to said base, said vertical guide rods adapted for slidably and removably engaging a multi-pin specimen holder comprising a pin base and a plurality of pins.

6. The biological tissue slicer system of claim 5 and wherein said vertical guide rods are movably engaged to one or more horizontal guide rods attached to said base, said horizontal guide rods being adapted to move the vertical guide rods in a horizontal direction.

7. The biological tissue slicer system of claim 6 wherein said one or more vertical guide rods are slidably engaged with said one or more horizontal guide rods via a U-channel, said U-channel having a top surface fixedly connected to said vertical guide rods, and having two side surfaces slidably engaging said horizontal guide rods.

8. The biological tissue slicer system of claim 7 wherein a rod is fixedly connected to said U-channel for moving said U-channel in a horizontal direction along said horizontal guide rods.

9. The biological tissue slicer system of claim 1 further comprising a multi-pin specimen holder, said multi-pin specimen holder comprising a pin base having a plurality of pins extending therefrom.

10. The biological tissue slicer system of claim 9 wherein said multi-pin specimen holder is removably attached to one or more vertical guide rods.

11. The biological tissue slicer system of claim 9 wherein said pins are arranged in one or more rows of pin, such that the pins in one row are spaced at a different distance than said pins in another row.

12. The biological tissue slicer system of claim 9 wherein said pins comprising said plurality of pins do not all have the same length or diameter.

13. The biological tissue slicer system of claim 1 wherein said base comprises a pivot rod attached to said base, and further comprising a plurality of pins pivotable about a pivot axis defined by said pivot rod.

14. The biological tissue slicer system of claim 13 further comprising at least one secondary pin, and wherein at least one of said plurality of pins has a hole for receiving said secondary pin through said hole.

15. The biological tissue slicer system of claim 1 further comprising a multi-pin specimen holder, one or more vertical guide rods, and one or more horizontal guide rods;
wherein said multi-pin specimen holder comprises a pin base and a plurality of pins for holding a tissue specimen;
wherein said multi-pin specimen holder is slidably engaged with said one or more vertical guide rods connected to said base;
wherein said one or more vertical guide rods are adapted to guide said multi-pin specimen holder in a vertical direction;
wherein said one or more vertical guide rods are slidably engaged with said one or more horizontal guide rods to move said vertical guide rods in a horizontal direction; and
wherein said base comprises a plurality of angled plates, each angled plate having a vertical member and a horizontal member, said plates configured to form said plurality of openings comprised of gaps between said plates.

16. The biological tissue slicer system of claim 15 wherein said blade cartridge having said plurality of blades is driven by a reciprocating driver, wherein said plurality of blades are configured to traverse said plurality of gaps between said plurality of angled plates such that the plurality of blades are capable of extending through and beyond the entire thickness of said tissue specimen.

17. The biological tissue slicer system of claim 1,
wherein said base comprises a plurality of plates aligned in parallel such that a gap between two of said plurality of plates forms one of said plurality of openings for permitting a blade of said plurality of blades to extend therethrough;
wherein said base is slidably engaged to a frame to move said base in a horizontal plane;
wherein said base comprises a pivot rod, and a plurality of pins pivotable about a pivot axis defined by said pivot rod; and
wherein at least one of said plurality of pins has a hole for receiving a secondary pin through said hole.

18. The biological tissue slicer system of claim 1 wherein said base comprises a pin base having a plurality of pins extending therefrom, said pins configured to form plurality of openings for permitting said blades to extend through said entire thickness of said tissue specimen.

19. The biological tissue slicer system of claim 1 wherein said blade cartridge with said plurality of blades is connected to a dual pivoting member, said dual pivoting member being pivotable about a first fixed pivot axis and a second movable pivot axis, and wherein said dual pivoting member is connected to said base.

20. A biological tissue slicer for slicing a biological tissue specimen comprising:
a base for holding said tissue specimen;
a vertical member attached to said base;
a blade cartridge having a plurality of blades for cutting said tissue specimen; and
a dual pivoting member having a first end pivotally and fixedly connected to said vertical member about a first fixed pivot axis, said dual pivoting member having a second end pivotally and movably connected to said blade cartridge about a second movable pivot axis such that said blades are adapted to cut said tissue specimen by moving both vertically and horizontally.

21. The biological tissue slicer of claim 20 where said base comprises a horizontal pin base having a plurality of pins extending therefrom, said pins adapted to receive a biological tissue specimen.

22. The biological tissue slicer of claim 21 wherein said pins are about 0.3 cm to 2 cm long.

23. The biological tissue slicer of claim 21 wherein said plurality of pins is affixed in a pin base configured to be removably inserted into a base plate.

24. The biological tissue slicer of claim 21 wherein said plurality of pins are spaced equidistantly from one another.

25. The biological tissue slicer of claim 20 wherein two vertical members are attached to said base and a transverse horizontal bar extends between said two vertical members, said dual pivoting member being pivotally and fixedly connected to said vertical members via said transverse bar to form said first fixed pivot axis.

26. The biological tissue slicer of claim 25 wherein said dual pivoting member is pivotably and movably connected to said blade cartridge via one or more holding brackets attached to a cartridge plate adapted to receive said blade cartridge.

27. The biological tissue slicer of claim 26 wherein said blade cartridge is removably positioned in said cartridge plate.

28. A biological tissue slicer for slicing a biological tissue specimen comprising:
a base for positioning a tissue specimen;
a plurality of pivoting pins pivotally attached to said base for engaging and holding said specimen on said base.

29. The biological tissue slicer of claim 28 wherein said base has a plurality of openings for permitting a plurality of blades to extend through the entire thickness of said tissue specimen.

30. The biological tissue slicer of claim 28 wherein said plurality of pins have a plurality of corresponding gaps therebetween.

31. The biological tissue slicer of claim 28 further comprising at least one secondary pin, and wherein at least one of said plurality of pins has hole for receiving said secondary pin.

32. The biological tissue slicer of claim 28 wherein said plurality of pins are J-shaped or U-shaped.

33. The biological tissue slicer of claim 28 wherein said base comprises a plurality of plates aligned in parallel such that a gap between two of said plurality of plates forms an opening for permitting a blade to extend therethrough.

34. The biological tissue slicer of claim 28 wherein said base is adjustably engaged to a frame.

35. The biological tissue slicer of claim 34 wherein said base has an upper rail for engaging a lower rail on said frame.
